(19) United States
(12) Patent Application Publication
(10) Pub. No.: US 2010/0049308 A1
(43) Pub. Date: Feb. 25, 2010

(54) VESSEL STENT WITH MULTI DRUG-COATINGS

(75) Inventor: Zhanjiang Yu, Beijing (CN)

Correspondence Address: OSHA LIANG L.L.P., TWO HOUSTON CENTER, 909 FANNIN, SUITE 3500, HOUSTON, TX, 77010, US

(73) Assignee: LEPU MEDICAL TECHNOLOGY (BEIJING) CO., LTD., Beijing (CN)

(21) Appl. No.: 12/515,044

(22) PCT Filed: Oct. 16, 2007
(86) PCT No.: PCT/CN2007/002964

§ 371 Date: May 15, 2009

(30) Foreign Application Priority Data
Nov. 20, 2006 (CN) 200620148126.4
Dec. 28, 2006 (CN) 200620166479.7

(51) Int. Cl. A61F 2/06

(52) U.S. Cl. 623/1.42

(57) ABSTRACT

A stent with multi drug-coatings includes a stent body and active drugs. A portion of the surface or the entire surface of the stent body is covered with at least one layer of active drug coating. Use of such a stent not only can accelerate endothelialization of coronary, but also resist cell proliferation, resist the migration of smooth muscle cells, reduce the formation of thrombus and the inflammatory reaction of cells and recover the flexibility of vascular tissues. Multi drug-coatings can prevent the multiple phases of restenosis, resist the release of drugs at different phases of endothelial repair, and play a role in co-resisting vessel stent restenosis by various drugs. Well-composed multi drug-coatings make the coating area and coating layers of drug-eluting stent and drugs compatibility more reasonable, make the use of drug-eluting stent more secure, and produce better treatment effects.

### TECHNICAL FIELD

The present invention belongs to the field of medical devices and particularly relates to a vessel stent with multi drug-coatings covered with multiple layers of different drugs on the surface of the vessel stent or both the inside and outside surfaces of the vessel stent.

### BACKGROUND ART

Since Sigwart, etc. applied intravascular metal stent to coronary artery the first time in 1987, it provided a good way to treat diseases that block vessels. However, the vessel stent restenosis remains the main reason that influences the effect of percutaneous coronary interventional treatment (PCI). Research has confirmed that the main reason that causes the vessel in-stent restenosis is a series of reactions, including the formation of thrombus induced after stent or saccule hurts vessel, the inflammatory reaction of cells, the migration and proliferation of smooth muscle cells and the flexibility recovery of vascular tissue happening in the process of implanting vessel stent.

To overcome the vessel in-stent restenosis, drug-eluting stent has shown unparalleled superiority in anti-stenosis performance. The existing drug-eluting stent mainly composes of a metal stent body, a carrier of drugs and drugs. In general, the drugs for resisting the vessel in-stent restenosis are coated on the surface of stent with the help of the carrier of drugs. However, the drug-coatings are all single coating, which causes no breakthrough in multiple links including coating area coating layers and drug compatibility or prevention of the happening of the vessel in-stent restenosis, thereby can not effectively control the release of drugs at different phases of endothelial repair and can not bring about good effect of treatment.

### SUMMARY OF THE INVENTION

The object of the present invention is to provide a vessel stent with multi drug-coatings with more reasonable coating area and coating layers, drug compatibility, which can prevent the multiple links of the happening of restenosis, control the release of drugs at different phases of endothelial repair and has better effect of treatment.

The solution to achieve the object of the present invention is as follows:

A vessel stent with multi drug-coatings comprises a stent body and active drugs, characterized in that a portion of surface or whole surface of the stent body is covered with at least one kind and at least one layer of drug coating.

The active drugs is dissolved in the solution of biodegradable polymeric materials or non-biodegradable polymeric materials; the biodegradable polymeric materials comprise one of homopolymer or copolymer of glycolide, lactide and ε-caprolactone, and copolymer formed by coplymerizing one of homopolymer or copolymer of glycolide, lactide and ε-caprolactone with multi-functional group of amino acids, polylactic acid, chitin, chitosan and collagen; the non-biodegradable polymeric materials comprise poly butyl methacrylate, polyethylene vinyl acetate copolymer, ethylene or vinyl acetate copolymer, polypropylene or polyacrylonitrile copolymer, poly-ε caprolactone.

The active drugs may include anti-inflammatory immunosuppressive agents, anti-proliferative drugs, anti-cell migration drugs, endothelialization of coronary drugs and polypeptide drugs. The anti-inflammatory immunosuppressive agents include sirolimus, tacrolimus, everolimus, immunosuppressive agents ABT-578, dexamethasone and mizoribine; the anti-proliferative drugs include rapamycin, paclitaxel, actinomycin, angiopeptim, vincristine and their derivatives, statin drugs, 2-chlorodeoxyadenosin, arsenic trioxide (As2O3) and ribozyme; the anti-cell migration drugs include batimastat, halofuginone, C-protease inhibitor and probucol; the coronary endothelialization drugs include endothelial growth factor, estradiols, penicillamine cyclopeptides, monoclonal antibody CD133 and its fragments, monoclonal antibody CD31 and its fragments, monoclonal antibody CD34 and its fragments and nucleic acid drugs.

Monoclonal antibody CD34 and its fragments acting as coronary endothelialization drugs are arranged on the inside surface of the stent body and rapamycin acting as drug resistant to proliferation of smooth muscle cells r is arranged on the outside surface of the stent body (1). The rapamycin drug is dissolved in the acetone and tetrahydrofuran solution of the non-biodegradable polymeric materials including polybutyl methacrylate, polyethylene vinyl acetate copolymer their equally mixed mixture. The rapamycin drug may also be dissolved in the acetone and tetrahydrofuran solution of one of the biodegradable polymeric materials including homopolymer or copolymer of glycolide, lactide and ε-caprolactone, and copolymer formed by coplymerizing one of homopolymer or copolymer of glycolide, lactide and ε-caprolactone with multi-functional group of amino acids, polylactic acid, chitin, chitosan and collagen.

Rapamycin acting as drug resistant to proliferation of smooth muscle cells is arranged on the outside surface of the stent body and monoclonal antibody CD34 and its fragments acting as coronary endothelialization drugs may be arranged on the outside surface of the rapamycin. The said rapamycin is dissolved in the acetone and tetrahydrofuran solution of the non-biodegradable polymeric materials including polybutyl methacrylate, polyethylene vinyl acetate copolymer their equally mixed mixture, or dissolved in one of the biodegradable polymeric materials including homopolymer or copolymer of glycolide, lactide and ε-caprolactone, and copolymer formed by coplymerizing one of homopolymer or copolymer of glycolide, lactide and ε-caprolactone with multiple-functional group of amino acids, polylactic acid, chitin, chitosan and collagen. Same size holes with multiple crystal phase structure are prepared on the surface of the stent body by chemical corrosion, electrochemical corrosion, anodic oxidation, micro-arc oxidation or micro-arc nitridation.

The outside surface of the stent body with holes is covered with rapamycin acting as drug resistant to proliferation of smooth muscle cells. The said rapamycin drug is dissolved in the solution of biodegradable polymeric materials or non-biodegradable polymeric materials, or directly dissolved in organic solvents and then is coated on the outside surface of the stent body.

The inside surface of the stent body with holes is embedded with the drug that captures vascular endothelial progenitor cells and promotes the endothelialization of the surface of the stent.

The drug that captures vascular endothelial progenitor cells and promotes the endothelialization of the surface of the stent includes endothelial growth factor, estradiols, penicillamine cyclopeptides (cyclo-GpenGRGDSPCA), monoclonal antibody CD34 and its fragments, monoclonal antibody CD31 and its fragments, monoclonal antibody CD133 and its fragments and nucleic acid drugs. Preferably, the inside surface of the stent body 1 with holes 101 is embedded with monoclonal antibody CD34 and its fragments or penicillamine cyclopeptides 203.

The beneficial effects of the present invention include:

1. The surface of the bare stent is covered with multiple layers of different drugs or the inside and outside surfaces are covered with different drugs, which can not only speed up endothelialization of coronary, but also resist cell proliferation, resist the migration of smooth muscle cells, reduce the formation of thrombus and the inflammatory reaction of cells and recover the flexibility of vascular tissue.

2. Two or more kinds of drugs acting at different links are coated on the same stent, which play the role of co-resisting the vessel in-stent restenosis by multiple drugs and resist the different pathological phases of the in-stent restenosis.

3. Multi drug-coatings have good anti-inflammatory effect and can resist the proliferation of cells, resist the migration of smooth muscle cells, speed up endothelialization of coronary, prevent multiple links of the happening of restenosis and resist the release of drugs at different phases of endothelial repair.

4. Drugs resistant to proliferation of smooth muscle cells and coronary endothelialization drug antibody are coated on the stent at the same time and the well-combined drug coating can solve the problems of the vessel in-stent restenosis and the stent later period thrombus effectively, make the use more secure and bring better effect of treatment.

5. On the surface of the bare stent, same size holes with poly crystalline phases structure are prepared on the surface of the equipment body by chemical corrosion, electrochemical corrosion, anodic oxidation, micro-arc oxidation or micro-arc nitridation. The release of the drugs resistant to proliferation of smooth muscle cells on the outside surface can be controlled through the utilization of the size and depth of the holes. The coronary endothelialization drugs can be fixed through the utilization of the electrical property of the stent and the size and depth of the holes. The two drugs acting at the different links are assembled at a stent platform, through which the advantages of many kinds of drugs are shown and the problems of inflammatory and thrombus that may be induced by the carrier of prior drugs stent can be avoided at the same time.

### BRIEF DESCRIPTION OF DRAWINGS

FIG. 1 illustrates cross-sectional cutaway view of the structure diagram of example 1 of the invention;

FIG. 2 illustrates cross-sectional cutaway view of the structure diagram of example 2 of the invention;

FIG. 3 illustrates cross-sectional cutaway view of the structure diagram of example 3 of the invention;

FIG. 4 illustrates cross-sectional cutaway view of the structure diagram of example 4 of the invention;

FIG. 5 illustrates the scanning electron microscope view of the stent body with holes of the invention;

FIG. 6 illustrates the scanning electron microscope view of the entire stent of the invention.

### DETAILED DESCRIPTION

The following examples are used to describe the present invention rather than limit the scope of the present invention.

A vessel stent with multi drug-coatings including a stent body 1 and active drugs 2, and metal materials such as 316L stainless steel, cobalt based alloy and nitinol may be selected for the stent body 1. FIG. 6 is the vertical view of the stent body with holes of the present invention. On the stent body 1 there are holes formed through electrochemical corrosion and a portion of surface or whole surface of the stent body 1 is covered with one or more and at least one layer active drugs 2 coatings. The active drugs 2 are dissolved in solution of biodegradable polymeric materials or non-biodegradable polymeric materials, which is then coated on the stent body 1 through coating methods such as spraying, dip-coating, roller coating, brush coating, sputtering and plasma polymerization to form the drug-coatings.

The biodegradable polymeric materials comprise one of homopolymer or copolymer of glycolide, lactide and ε-caprolactone, and copolymer formed by coplymerizing one of homopolymer or copolymer of glycolide, lactide and ε-caprolactone with multi-functional group of amino acids, polylactic acid, chitin, chitosan and collagen.

The non-biodegradable polymeric materials comprise polybutyl methacrylate, polyethylene vinyl acetate copolymer, EVA, polypropylene or polyacrylonitrile copolymer, poly-ε caprolactone.

The active drugs 2 are selected from four kinds of active drugs advantageously resisting the vessel in-stent restenosis, one of which is immunosuppressive agents with anti-inflammatory such as sirolimus, tacrolimus, everolimus, immunosuppressive agents ABT-578, dexamethasone and mizoribine; the second kind of active drug is the anti-proliferative drug such as rapamycin, paclitaxel, actinomycin, angiopeptim, vincristine and its derivatives, arsenic trioxide (As2O3), statin drugs, 2-chlorodeoxyadenosin and ribozyme; the third kind of active drug is anti-cell migration drug such as batimastat, halofuginone, C-protease inhibitor and probucol; the fourth kind of active drug is the drug speeding up coronary endothelialization such as endothelial growth factor (VEGF), estradiols, monoclonal antibody CD34 and its fragments, monoclonal antibody CD133 and its fragments, monoclonal antibody CD31 and its fragments and nucleic acid drugs (DNA, RNA and RNAi). Research and clinical analysis have confirmed that rapamycin drug 202 is an T cell inhibitor, as a kind of good anti-cell proliferation drug it can prevent T cell from transforming from G₁ period to S period and prevent G_{O} period of B cell; monoclonal antibody CD34 and its fragments 201 can capture vascular endothelial progenitor cells (EPC) by combining antigen with antibody, speed up the differentiation of vascular endothelial progenitor cells on the surface of the stent into endothelial cells, and promote the repair of endothelium; for arginine-glycine-aspartic acid (RGD) cyclopeptides and modified products penicillamine cyclopeptides (cyclo-GpenGRGDSPCA) 203 that have higher affinity to integrin α5β1 receptor than to αvβ3 receptor, monoclonal antibody CD34 and its fragments 201 can capture more endothelial progenitor cells (EPCs) by combing the receptor with ligand to make the EPCs differentiate into vascular endothelial cells on the surface of the stent quickly and therefore promote the repair of endothelium.

Thus, the inside surface of the stent body 1 with holes 101 is embedded with monoclonal antibody CD34 and its fragments or arginine-glycine-aspartic acid (RGD) polypeptide drug 203, and the monoclonal antibody CD34 201 and its fragments or arginine-glycine-aspartic acid (RGD) polypeptide drug 203 can be dissolved in phosphate buffer (pH 7.2˜7.4) or carbonate buffer (pH 9.6) and then be directly embedded in the holes 101 on the inside surface of the stent body 1. The outside surface of the stent body 1 is sprayed with drug 202 resistant to proliferation of smooth muscle cells, which can be sprayed with the help of non-biodegradable polymeric materials or biodegradable polymeric materials or being dissolved in tetrahydrofuran solution directly (the weight percent is 0.2˜5%).

Hereafter the preferable embodiments will be given:

### Example 1

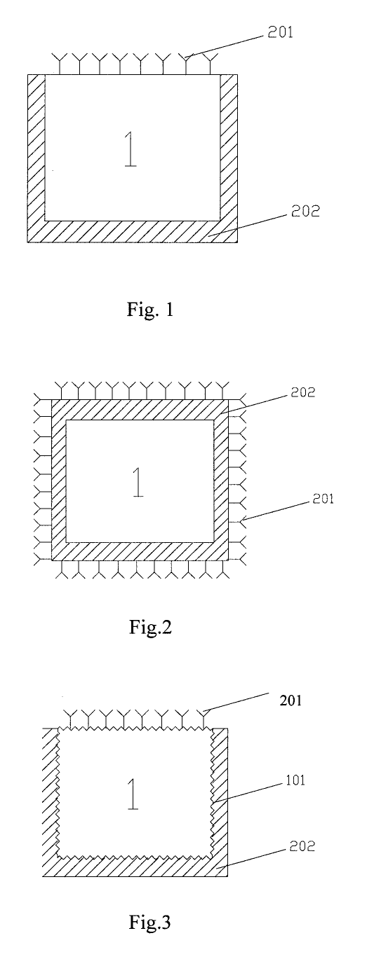

FIG. 1 illustrates cross-sectional cutaway view of the structure diagram of example 1 of the invention. The inside surface of the stent body 1 is covered with a layer of monoclonal antibody CD34 and its fragments 201, the outside of which is covered with rapamycin drug 202. The said rapamycin drug 202 is dissolved in the acetone and tetrahydrofuran solution of the non-biodegradable polymeric materials including polybutyl methacrylate PBMA, polyethylene vinyl acetate copolymer PEVA and their equally mixed mixture, or dissolved in the acetone and tetrahydrofuran solution of the biodegradable polylactic acid or glycolic acid copolymer PLGA, or polylactic acid, and then sprayed or dig-coated on the outside surface of the stent body 1.

### Example 2

FIG. 2 illustrates cross-sectional cutaway view of the structure diagram of example 2 of the Invention. The whole surface of the stent body 1 is covered with a layer of rapamycin drug 202. On the outside surface of rapamycin drug 202 is covered with a layer of monoclonal antibody CD34 and its fragment drug 201. The rapamycin drug 202 is dissolved in the tetrahydrofuran solution of the non-biodegradable polybutyl methacrylate PBMA or the biodegradable polylactic acid or glycolic acid copolymer PLGA, and then sprayed or dig-coated on the outside surface of the stent body 1.

### Example 3

FIG. 3 illustrates cross-sectional cutaway view of the structure diagram of example 3 of the Invention. The inside surface of the stent body 1 with holes 101 is embedded with monoclonal antibody CD34 and its fragment drug 201, the outside surface of which is covered with rapamycin 202 acting as drug resistant to proliferation of smooth muscle cells, rapamycin may be dissolved in the non-biodegradable polybutyl methacrylate of 0.2-5% (weight percent), or tetrahydrofuran solution of the biodegradable polylactic acid-polyglycolic acid copolymer of 0.5-10% (weight percent), or directly dissolved in the tetrahydrofuran solution of 0.5-10% (weight percent), and then sprayed on the outside surface of the stent body 1.

### Example 4

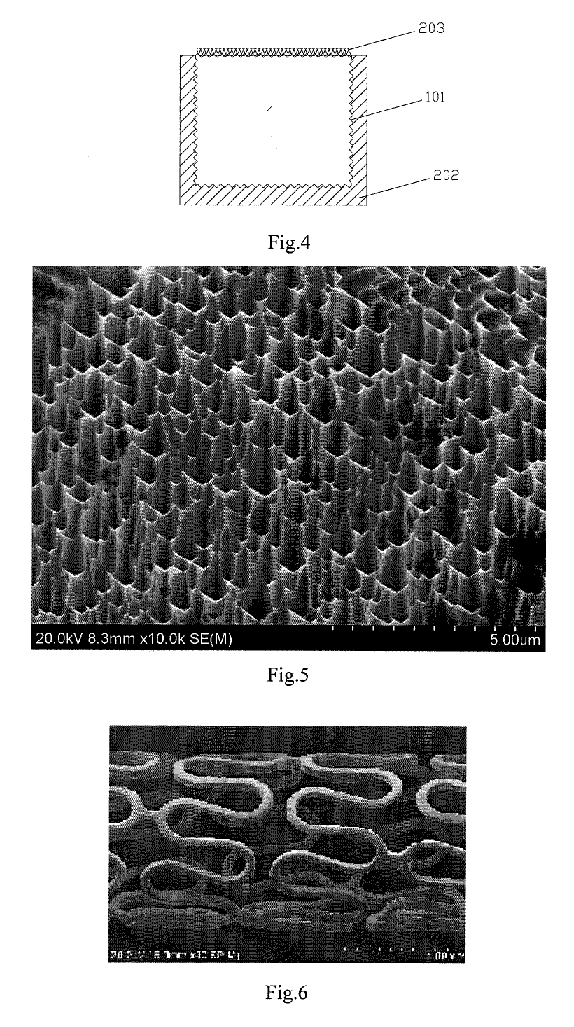

FIG. 4 illustrates cross-sectional cutaway view of the structure of the example 4 of the invention. The inside surface of the stent body 1 with holes 101 is embedded with arginine-glycine-aspartic acid (RGD) polypeptide drug 203. The polypeptide drug 203 is dissolved in phosphate buffer (pH 7.2˜7.4) or carbonate buffer (pH 9.6) and then embedded on the inside surface of the stent body 1. The outside surface of the stent body 1 is covered with rapamycin 202 acting as drug resistant to proliferation of smooth muscle cells which may be dissolved in the solution of non-biodegradable 1 polybutyl methacrylate of 0.2-5% (weight percent) or the tetrahydrofuran solution of biodegradable polylactic acid-polyglycolic acid copolymer of 0.5-10% (weight percent), or directly dissolved in the tetrahydrofuran solution of 0.5-10% (weight percent), and then sprayed on the outside surface of the stent body 1.

While the invention has been described in terms of various specific embodiments and general description, it will be obvious to those skilled in the art that certain modification or improvement should be made to the invention as described without departing from the scope of the invention.

### INDUSTRIAL APPLICABILITY

Basen on the prior vessel stent, the present invention utilizes physical or chemical method to corrode and form holes. According to their properties, the active drugs are dissolved in the solution of biodegradable polymeric materials or non-biodegradable polymeric materials, or directly dissolved in organic solvents (tetrahydrofuran, acetone, chloroform, etc.) or phosphate buffer (pH 7.2˜7.4) or carbonate buffer (pH 9.6), which can be coated on the stent body through drug coating methods such as spraying, dip-coating, roller coating, brush coating, sputtering and plasma polymerization to form the drug-coatings. The active drug monoclonal antibody CD34 and its fragments can capture vascular endothelial progenitor cells by combing antigen with antibody, speed up the differentiation of vascular endothelial progenitor cells on the surface of the stent into endothelial cells and promote the repair of endothelium. As a good anti-cell proliferation drug, the active drug, rapamycin can prevent T cell from transforming from G₁ period to S period and prevent G_{O} period of B cell; arginine-glycine-aspartic acid (RGD) cyclopeptides and its modified products-penicillamine cyclopeptides (cyclo-GpenGRGDSPCA) can capture more endothelial progenitor cells (EPCs) by combing the receptor with ligand to make the EPCs differentiate into vascular endothelial cells on the surface of the stent quickly and therefore promote the repair of endothelium. Thus, by the way of embedding the drugs mentioned above on the surface of the stent body with holes and utilizing the size and depth, the speed of release of drugs can be effectively controlled. At the same time, the coronary endothelialization drugs can be fixed through the utilization of the electrical property of the stent and the size and depth of the holes. The two drugs acting at the different links are assembled at a stent platform, through which the advantages of many kinds of drugs are shown, the problems of inflammatory and thrombus that may be induced by the carrier of prior drugs stent can be avoided, and the rate of formation of thrombus and the incidence of the disease of in-stent restenosis can be reduced effectively at the same time.

1. A vessel stent with multi drug-coatings includes a stent body and active drugs, characterized in that, a portion of surface or whole surface of the stent body is covered with at least one kind and at least one layer of active drugs coating.

2. The vessel stent with multi drug-coatings according to claim 1, characterized in that, the active drugs are dissolved in the solution of biodegradable or non-biodegradable polymeric materials; the said biodegradable polymeric materials comprise one of homopolymer or copolymer of glycolide, lactide and ε-caprolactone, and copolymer formed by coplymerizing one of homopolymer or copolymer of glycolide, lactide and ε-caprolactone with multi-functional group of amino acids, polylactic acid, chitin, chitosan and collagen; the said non-biodegradable polymeric materials comprise polybutyl methacrylate, polyethylene vinyl acetate copolymer, ethylene or vinyl acetate copolymer, polypropylene or polyacrylonitrile copolymer, poly-ε caprolactone.

3. The vessel stent with multi drug-coatings according to claim 1, characterized in that, the active drugs include anti-inflammatory immunosuppressive agents, anti-proliferative drugs, anti-cell migration drugs, endothelialization of coronary drugs and polypeptide drugs; the said anti-inflammatory immunosuppressive agents include sirolimus, tacrolimus, everolimus, immunosuppressive agents ABT-578, dexamethasone and mizoribine; the anti-proliferative drugs include rapamycin, paclitaxel, actinomycin, angiopeptim, vincristine and their derivatives, statins drugs, 2-chlorodeoxyadenosin, arsenic trioxide (As₂O₃) and ribozyme; the anti-cell migration drugs include batimastat, halofuginone, C-protease inhibitor and probucol; the coronary endothelialization drugs include endothelial growth factor, estradiols, penicillamine cyclopeptides, monoclonal antibody CD133 and its fragments, monoclonal antibody CD31 and its fragments, monoclonal antibody CD34 and its fragments and nucleic acid drugs.

4. The vessel stent with multi drug-coatings according to claim 1, characterized in that, monoclonal antibody CD34 and its fragments acting as coronary endothelialization drugs are arranged on the inside surface of the stent body and drugs resistant to proliferation of smooth muscle cells is arranged on the outside surface of the stent body; the said rapamycin drug is dissolved in the acetone and tetrahydrofuran solution of the non-biodegradable polymeric materials including polybutyl methacrylate, polyethylene vinyl acetate copolymerand their equally mixed mixture, or dissolved in one of the biodegradable polymeric materials including homopolymer or copolymer of glycolide, lactide and ε-caprolactone, and copolymer formed by coplymerizing one of homopolymer or copolymer of glycolide, lactide and ε-caprolactone with multi-functional group of amino acids, polylactic acid, chitin, chitosan and collagen.

5. The vessel stent with multi drug-coatings according to claim 1, characterized in that, rapamycin acting as a drug resistant to proliferation of smooth muscle cells is arranged on the outside surface of the stent body and monoclonal antibody CD34 and its fragments acting as coronary endothelialization drugs are arranged on the outside surface of the said; rapamycin is dissolved in the acetone and tetrahydrofuran solution of the non-biodegradable polymeric materials including polybutyl methacrylate, polyethylene vinyl acetate copolymer and their equally mixed mixture, or dissolved in one of the biodegradable polymeric materials including homopolymer or copolymer of glycolide, lactide and ε-caprolactone, and copolymer formed by coplymerizing one of homopolymer or copolymer of glycolide, lactide and ε-caprolactone with multi-functional group of amino acids, polylactic acid, chitin, chitosan and collagen.

6. The vessel stent with multi drug-coatings according to claim 1, characterized in that, same size holes with multicrystal phase structure are prepared on the surface of the stent body by chemical corrosion, electrochemical corrosion, anodic oxidation, micro-arc oxidation or micro-arc nitridation.

7. he vessel stent with multi drug-coatings according to claim 6, characterized in that, the outside surface of the stent body with holes is covered with rapamycin acting as drug resistant to proliferation of smooth muscle cells; rapamycin is dissolved in the solution of biodegradable or non-biodegradable polymeric materials, or directly dissolved in organic solvents and then is coated on the outside surface of the stent body.

8. The vessel stent with multi drug-coatings according to claim 6, characterized in that, the inside surface of the stent body with holes is embedded with drugs that capture vascular endothelial progenitor cells and promote the endothelialization of the the stent surface.

9. The vessel stent with multi drug-coatings according to claim 8, characterized in that, the drugs that capture vascular endothelial progenitor cells and promote the endothelialization of the the stent surface include endothelial growth factor, estradiols, penicillamine cyclopeptides, monoclonal antibody CD34 and its fragments, monoclonal antibody CD31 and its fragments, monoclonal antibody CD133 and its fragments and nucleic acid drugs.

10. The vessel stent with multi drug-coatings according to claim 9, characterized in that, the inside surface of the stent body with holes is embedded with monoclonal antibody CD34 and its fragments or penicillamine cyclopeptides.

11. The vessel stent with multi drug-coatings according to claim 2, characterized in that, monoclonal antibody CD34 and its fragments acting as coronary endothelialization drugs are arranged on the inside surface of the stent body and drugs resistant to proliferation of smooth muscle cells is arranged on the outside surface of the stent body; the said rapamycin drug is dissolved in the acetone and tetrahydrofuran solution of the non-biodegradable polymeric materials including polybutyl methacrylate, polyethylene vinyl acetate copolymerand their equally mixed mixture, or dissolved in one of the biodegradable polymeric materials including homopolymer or copolymer of glycolide, lactide and ε-caprolactone, and copolymer formed by coplymerizing one of homopolymer or copolymer of glycolide, lactide and ε-caprolactone with multi-functional group of amino acids, polylactic acid, chitin, chitosan and collagen.

12. The vessel stent with multi drug-coatings according to claim 3, characterized in that, monoclonal antibody CD34 and its fragments acting as coronary endothelialization drugs are arranged on the inside surface of the stent body and drugs resistant to proliferation of smooth muscle cells is arranged on the outside surface of the stent body; the said rapamycin drug is dissolved in the acetone and tetrahydrofuran solution of the non-biodegradable polymeric materials including polybutyl methacrylate, polyethylene vinyl acetate copolymerand their equally mixed mixture, or dissolved in one of the biodegradable polymeric materials including homopolymer or copolymer of glycolide, lactide and ε-caprolactone, and copolymer formed by coplymerizing one of homopolymer or copolymer of glycolide, lactide and ε-caprolactone with multi-functional group of amino acids, polylactic acid, chitin, chitosan and collagen.

13. The vessel stent with multi drug-coatings according to claim 2, characterized in that, rapamycin acting as a drug resistant to proliferation of smooth muscle cells is arranged on the outside surface of the stent body and monoclonal antibody CD34 and its fragments acting as coronary endothelialization drugs are arranged on the outside surface of the said; rapamycin is dissolved in the acetone and tetrahydrofuran solution of the non-biodegradable polymeric materials including polybutyl methacrylate, polyethylene vinyl acetate copolymer and their equally mixed mixture, or dissolved in one of the biodegradable polymeric materials including homopolymer or copolymer of glycolide, lactide and ε-caprolactone, and copolymer formed by coplymerizing one of homopolymer or copolymer of glycolide, lactide and ε-caprolactone with multi-functional group of amino acids, polylactic acid, chitin, chitosan and collagen.

14. The vessel stent with multi drug-coatings according to claim 3, characterized in that, rapamycin acting as a drug resistant to proliferation of smooth muscle cells is arranged on the outside surface of the stent body and monoclonal antibody CD34 and its fragments acting as coronary endothelialization drugs are arranged on the outside surface of the said; rapamycin is dissolved in the acetone and tetrahydrofuran solution of the non-biodegradable polymeric materials including polybutyl methacrylate, polyethylene vinyl acetate copolymer and their equally mixed mixture, or dissolved in one of the biodegradable polymeric materials including homopolymer or copolymer of glycolide, lactide and ε-caprolactone, and copolymer formed by coplymerizing one of homopolymer or copolymer of glycolide, lactide and ε-caprolactone with multi-functional group of amino acids, polylactic acid, chitin, chitosan and collagen.
